(19) United States
(12) Patent Application Publication
(10) Pub. No.: US 2018/0078402 A1
(43) Pub. Date: Mar. 22, 2018

(54) STOMA TUBE METHOD AND APPARATUS

(71) Applicant: MELINDA ERICSON, GENTRY, AR (US)

(72) Inventor: MELINDA ERICSON, GENTRY, AR (US)

(21) Appl. No.: 15/824,774

(22) Filed: Nov. 28, 2017

(51) Int. Cl. A61F 5/445; A61F 5/44; A61B 17/34

(52) U.S. Cl. CPC A61F 5/445; A61M 2202/068; A61B 17/3415; A61F 5/4404

(57) ABSTRACT

A stoma tube apparatus and method of use are presented herein. The apparatus includes a flexible elongated body having a first end and a second end. The body is hollow along its length. A rib is formed along an outer surface of the body to provide increased strength. A plurality of apertures pass through the outer surface of the tube so as to permit the passage of fluid and fecal matter into the hollow portion of the tube for drainage. A barrier ring is used to limit undesired movement of the tube in relation to the patient's body. In use, one or more medical devices may be coupled to a first end of the elongated body to facilitate extraction of the fecal matter through the apertures.

### BACKGROUND

### 1. Field of the Invention

The present application relates to a medical device, and more particularly to an improved stoma tube for use with surgically created openings in the body for accessing the intestines so as to allow fecal matter to pass through a plurality of perforations in the tube.

### 2. Description of Related Art

Approximately one million Americans suffer from such significant intestinal issues such that it has required an operation. Common examples of operations would include a colostomy or an ileostomy. The operation bypasses some or all of their intestines by the creation of a stoma, or a surgically created opening in the body. Even more people in the United States have required temporary stomas in order to deal with a variety of medical issues. While fairly routine as a consequence of its frequent usage, the process of maintaining these stomas has historically been complicated, cumbersome, and often dangerous due to frequent infections, stenosis/stricture (narrowing), and even blockages, all of which can require repeated surgeries and follow up procedures. Stenosis and stricture alone affect ten percent of all stoma patients, with half of those ten percent being affected in the first year. And those with stoma treatments in emergency room contexts are typically sent home without any tubes or follow up treatment options other than the hope that the initial attempt to dissolve the blockage has been successful. Present methods and devices fail to provide adequate care and treatment.

Although strides have been made to provide treatment options to patients, shortcomings remain. It is desired that an apparatus be developed for improving the quality of life for stoma patients and also reducing the risks of future infections, stenosis, blockages, and additional surgeries.

### SUMMARY OF THE INVENTION

It is an object of the present application to provide a stoma tube apparatus that is useful in improving the quality of life for stoma patients and also reducing the risks of future infections, stenosis, blockages, and additional surgeries. The apparatus of the present application is designed and configured for use within the intestines of patients. The apparatus is configured to include a plurality of perforations in the tube to allow for the passage of fecal matter there through.

It is a further object of the present application that the assembly be further strengthened at the tip for improved performance and ease of use when initially inserting the tube into the stoma. Along the same lines, an added strengthening rib is included along the length of the tube to improve rigidity as the tube is located within the intestine.

Furthermore, it is an object of the present application to provide a device for the securing of the tube within the body. A ring is provided to selectively grip or attach to the tube at any location and act to contact the patient around the stoma so as to prevent the undesired slippage of the tube inside the patient.

The method of using the assembly is simplified in that a user merely has to locate the tube within the body through the stoma to permit drainage of the intestine. The perforations or holes along the length of the tube increase the ability for the fecal matter to pass through and alleviate any blockages. Use of this apparatus reduces the risks of future infections, stenosis, blockages, and additional surgeries. In this way, this apparatus overcomes the disadvantages inherent in the prior art.

The more important features of the apparatus have thus been outlined in order that the more detailed description that follows may be better understood and to ensure that the present contribution to the art is appreciated. Additional features of the apparatus will be described hereinafter and will form the subject matter of the claims that follow.

Many objects of the present assembly will appear from the following description and appended claims, reference being made to the accompanying drawings forming a part of this specification wherein like reference characters designate corresponding parts in the several views.

Before explaining at least one embodiment of the system in detail, it is to be understood that the assembly is not limited in its application to the details of construction and the arrangements of the components set forth in the following description or illustrated in the drawings. The apparatus is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present apparatus. It is important, therefore, that the claims be regarded as including such equivalent constructions insofar as they do not depart from the spirit and scope of the present apparatus.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a stoma tube apparatus according to an embodiment of the present application.

FIG. 2 is a front view of a human body showing a stoma for use with the stoma tube apparatus of FIG. 1.

FIG. 3 is a partial side section view of the stoma tube apparatus of FIG. 1 passing into the stoma.

FIG. 4 is a partial side section view of stoma tube apparatus of FIG. 3 further set within the stoma.

While the apparatus and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the apparatus described herein may be oriented in any desired direction.

The apparatus and method in accordance with the present application overcomes one or more of the above-discussed problems commonly associated with elevated platforms discussed previously. In particular, the apparatus is configured to provide a plurality of apertures or perforations within the body of the tube to permit the passage of liquid and fecal matter to enter inside the hollowed tube for drainage. The apparatus is configured to selectively locate a ring around the outside of the tube designed to adapt to the depth of penetration of the tube through the stoma and stabilize the tube depth as the patient moves. These and other unique features of the apparatus are discussed below and illustrated in the accompanying drawings.

The apparatus and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The apparatus and method of the present application is illustrated in the associated drawings. The apparatus includes a flexible elongated body having a first end and a second end. The body is hollow along its length. A rib is formed along an outer surface of the body to provide increased strength. A plurality of apertures pass through the outer surface of the tube so as to permit the passage of fluid and fecal matter into the hollow portion of the tube for drainage. Additional features and functions of the apparatus are illustrated and discussed below.

Referring now to the Figures wherein like reference characters identify corresponding or similar elements in form and function throughout the several views. The following Figures describe the apparatus of the present application and its associated features. With reference now to the Figures, an embodiment of the stoma tube apparatus of use are herein described. It should be noted that the articles “a”, “an”, and “the”, as used in this specification, include plural referents unless the content clearly dictates otherwise.

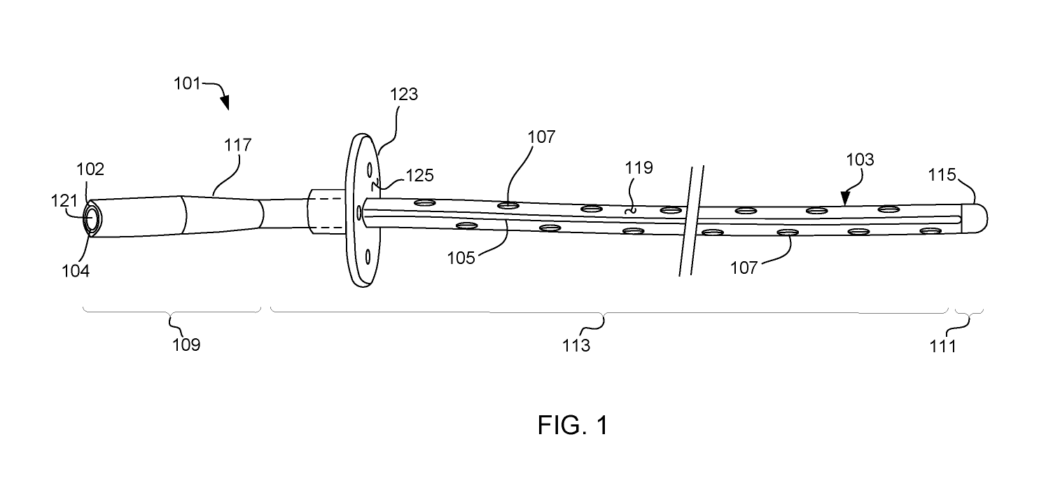

Referring now to FIG. 1 in the drawings, a perspective view of a stoma tube apparatus 101 is illustrated. Tube 101 includes a body 103, a rib 105, and a plurality of apertures 107 formed along a portion of body 103. The apertures 107 allow tube 101 to pass solid fecal matter as well as liquid into the hollowed center of body 103 so that such matter is drained from the intestines of a patient. This improves the quality of life for stoma patients and also reduces the risks of future infections, stenosis, blockages, and additional surgeries.

Body 103 is a flexible material that is shaped in a relatively cylindrical form. Body 105 is hollowed to allow for substances such as solids and liquids to pass internally therein. Body 103 is configured to bend and flex so as to easily pass through the stoma and into the intestines without much resistance. Body 103 may be made from one or more layers 102/104 of material. The layering of material may affect the flexibility of the body to some degree. A common type of material may be a silicone based material. Due to its inclusion into a living body, body 103 is sterile and safe for internal use. It is understood that each layer may include different performance characteristics, such as hardness, elasticity, and permeability. In fact, it is desired that various portions of body 103 in fact have different flexibilities.

Body 103 includes three sections, a first end 109, a second end 111, and a center portion 113. As seen in FIG. 1 and the other Figures the length of body 103 is not particularly restricted to any length. For purposes of seeing both ends 109/111 of body 105, center portion 113 was visually cut.

At second end 111, body 103 includes a tip 115 configured to enclose the hollowed center 121 of body 105. Tip 115 is hardened and more rigid compared to center portion 113. This may be due to material differences and/or layering differences there between them. Tip 115 extends partially down the length of body 103 (i.e. 1 inch). The increases rigidity of tip 115 allows for better entry into the stoma as body 103 is less likely to compress or deflect when pushed. Tip 115 does not include apertures 107. Additionally, as body 103 passes within the intestines, the lack of a hole in tip 115 helps to avoid issues that may arise from abrasions of internal walls of the intestine.

First end 109 is defined as the portion of body 103 that flares outward and increases in diameter compared to that of either or both of center portion 113 and second end 111. End 109 includes a tapered portion 117 where the diameter slowly increases in size followed by a constant diameter reaching to the end of body 103. End 109 is used to permit coupling to medical devices to aid in the extraction of fecal matter and liquid from the intestines. Some examples of medical devices may be slip tip syringes and bags. As with second end 111, first end 109 may include different layering and materials than that found in center portion 113, which may influence performance characteristics similarly.

As noted previously with respect to tip 115, selected portions of apparatus 101 may be integrated or modified to permit decreased flexibility. For example, apparatus 101 further includes a rib 105 that acts similarly to that of tip 115. Rib 105 is configured to extend along at least a portion of an outer surface 119 of center portion 113. Rib 105 is pictured as extending along the length of body 103 from tip 115 to just prior to first end 109. Rib 105 is also formed on an opposing side (not seen) of body 103. Rib 105 acts to partially limit flexibility of body 103 in selected directions to aid in inserting apparatus 101 into the body. For example, flexure perpendicular to a plane defined by two ribs is simpler than flexure parallel to a plane defined by two ribs. One or more ribs 105 may be used to restrict and control the direction of flexure of body 103. Rib 105 may be made from similar materials as body 103 and is integrally formed therein. One or more layers 102/104 may be used to create the external protrusion that forms rib 105.

Body 103 further includes a plurality of apertures 107 configured to extend from outer surface 119 to a hollowed center 121 of body 103. Apertures 107 are configured in size and shape to permit the passage of fecal matter, air, and fluid to enter into hollowed center 121 and subsequently pass out of the stoma for discharging. Inclusion of apertures 107 along the length of center portion 113 increases the volume of matter that may be discharged and the likelihood that apparatus 101 will be positioned in a manner to accept the fecal matter. The number and shape of the apertures 107 are not limited herein to those depicted in the Figures. The representations are merely exemplary in nature and other shapes and sizes are possible and considered within the scope of the present application. Of note is that the overall shape and sizing of apertures 107 should not work to impede the function of apparatus 101 in being usable for insertion. For example, if too many apertures 107 are used then the strength of body 103 may be compromised making it ineffective in either inserting into the patient or in that the hollowed center possibly collapses within the patient. The routing of ribs 105 may be altered to address the size, shape, and placement of apertures 107.

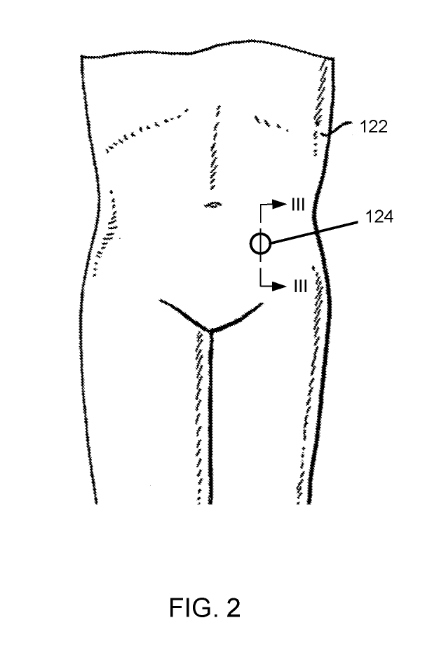

Referring now also to FIG. 2 in the drawings, a front view of a patient/human body 122 with a stoma 124 is illustrated. Apparatus 101 may further include a barrier ring 123 that extends around outer surface 119. Ring 123 is configured to translate along body 103 around at least center portion 113. Ring 123 is configured to grip outer surface 119 through interference fit/friction. Application of gentle pressure by a user can cause translation of ring 123 along the length of body 103. In use, ring 123 is configured to make contact with a portion of the patient 122 around the stoma 124. Front surface 125 may contact body 122 directly or a material in communication with body 122 adjacent stoma 124. Ring 123 is configured to prevent undesired movement of body 103 within stoma 124. Additionally, ring 123 allows cleaning without disruption of body 103 in intestines. Ring 123 may be separated from body 103 at selected times.

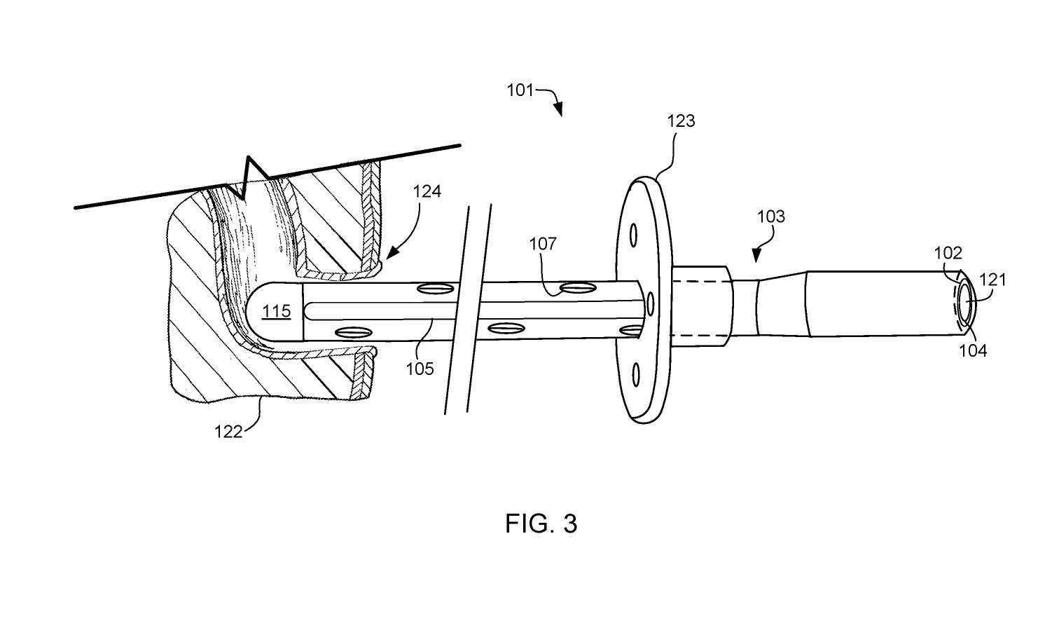

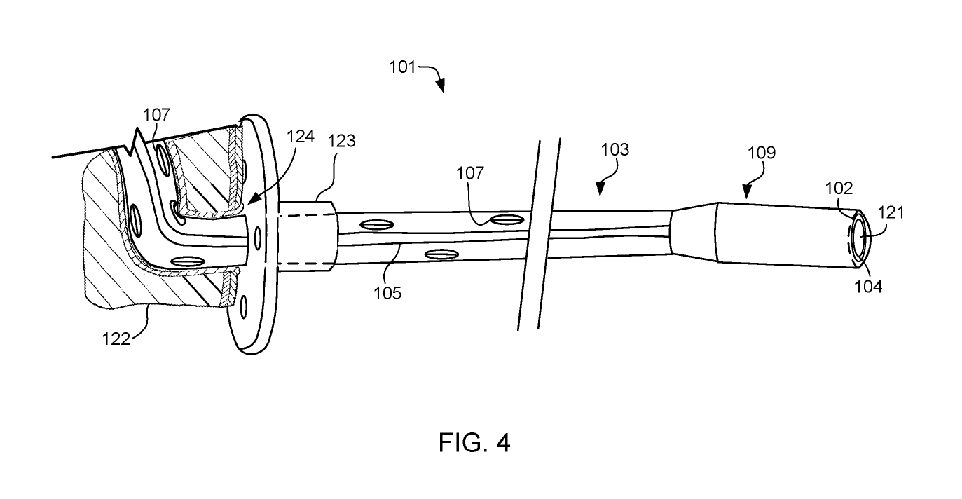

Referring now also to FIGS. 3 and 4 in the drawings, enlarged partial side section views of stoma 124 are provided with apparatus 101 inserted therein to differing amounts of length. As seen in FIG. 3, second end 111 is inserted into stoma 124. A user grips body 103 and presses tip 115 into stoma 124. Tip 115 proceeds to pass further into the patient 122 with the application of force applied by the user. As seen in FIG. 4, body 103 is clearly within body 122. Barrier ring 123 is pictured adjacent to stoma 124 for exemplary purposes. It is a purpose of apparatus 101 to facilitate drainage of fecal matter out first end 109. If apertures 107 are left outside stoma 124 upon extraction of the fecal matter, the fecal matter would be spilled. This highlights the importance in using a particular apparatus length and/or aperture pattern and location that works for the particular need of patient 122. Body 103 may be further pushed inside stoma 124 until apertures 107 are located internally within patient body 122. One or more medical devices may be coupled to first end 109 as needed to facilitate drainage of fecal matter. Additionally, the location of the barrier ring 123 may be adjusted as needed for the patient 122.

The current application has many advantages over the prior art including at least the following: (1) varied levels of flexibility throughout the length of the flexible body with the inclusion of a rib; (2) a plurality of apertures distributed along the length of the flexible body to permit the discharge and drainage of fecal matter and liquid from the intestine; (3) a strengthened tip to ease insertion through the stoma; (4) a barrier ring to hold the location of the flexible body at a particular depth within the patient body; and (5) flared out first end to communicate with other medical devices to aid in extracting the fecal matter.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A tube apparatus, comprising:
a flexible elongated body having a first end and a second end, the body being hollowed along its length;
a rib formed along an outer surface of the body; and
a plurality of apertures passing through the outer surface of the tube so as to permit passage of fluid and fecal matter into the hollowed portion of the tube for drainage.

2. The apparatus of claim 1, wherein the body is flared outward at the first end such that the diameter of the first end is greater than the second end.

3. The apparatus of claim 1, wherein the body includes a plurality of different material layers.

4. The apparatus of claim 1, wherein the second end has an enclosed tip.

5. The apparatus of claim 4, wherein the tip is hardened so as to minimize flexure of the tip.

6. The apparatus of claim 4, wherein the tip is without any of the plurality of apertures.

7. The apparatus of claim 1, wherein the plurality of apertures are limited to the area of the body between the first end and the second end.

8. The apparatus of claim 1, wherein the flexibility of the body varies along its length.

9. The apparatus of claim 1, further comprising:
a barrier ring in communication with the outer surface of the body.

10. The apparatus of claim 9, wherein the barrier ring is configured to selectively translate along the outer surface.

11. The apparatus of claim 9, wherein friction between the outer surface and the barrier ring restrict movement of the barrier ring.

12. The apparatus of claim 9, wherein the barrier ring prevents undesired movement of the body relative to a patient.

13. A method of treating a patient through a stoma, comprising:
obtaining a flexible elongated body having a first end and a second end, the body including:
a hollowed center along its length;
a rib formed along an outer surface of the body; and
a plurality of apertures passing through the outer surface of the tube so as to permit passage of fluid and fecal matter into the hollowed portion of the tube for drainage;
inserting the body through the stoma;
adjusting the location of a barrier ring along the outer surface of the body so as to contact the patient and restrict undesired movement within the stoma; and
draining fecal matter and liquid through the plurality of apertures through the hollowed center.

14. The apparatus of claim 13, wherein friction between the outer surface and the barrier ring restrict movement of the barrier ring.

15. The apparatus of claim 13, wherein the plurality of apertures are limited to the area of the body between the first end and the second end.
